(19) United States
(12) Patent Application Publication
(10) Pub. No.: US 2007/0065375 A1
(43) Pub. Date: Mar. 22, 2007

(54) Oral composition comprising micron-sized lanthanide metal oxide

(76) Inventor: Matthew Jonathan Pickles, Bebington Merseyside (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/551,012

(22) PCT Filed: Mar. 24, 2004
(86) PCT No.: PCT/EP04/03234

§ 371 Date: Jun. 27, 2006

(30) Foreign Application Priority Data
Apr. 2, 2003 (EP) 03252083.5

(51) Int. Cl. A61K 8/19

(52) U.S. Cl. 424/49; 424/617

(57) ABSTRACT

Oral composition comprising as abrasive a mixture of a lanthanide metal oxide and a further abrasive, characterised in that the lanthanide metal oxide has a weight average particle size of from 0.5 to 50 ÿm.

The present invention relates to an oral care composition comprising an improved abrasive system.

U.S. Pat. No. 5,718,885 (Gingold) discloses the use of particles selected from compounds comprising metals from groups IB, IIA, IIB, IIIA, IIIB, IVB, VA, VIA, VIB, VIIB and VIIIB of the periodic table including the Lanthanides in an oral care composition for treating dentinal hypersensitivity. More preferably, the metals are selected from Y, Ce, AL and Zr, most preferably the metal is either Al or Zr.

The compounds are present in an aqueous colloid and must be capable of having a cationic charge in an aqueous environment. Suitably they are present as halides, silicates, acetates, oxides and hydroxides. Most preferably they are oxides. The compounds have a submicron particle size.

U.S. Pat. No. 3,573,886 (Goetzinger) discloses compositions for polishing and comprising rare-earth oxide and woolastonite. The rare-earth metal oxides disclosed are used in their naturally occurring state, i.e. comprising a mixture of metals. The compositions are used to grind glass.

DE 28 02 489 (Bayer) discloses cerium salt solutions for use in toothpastes. Cerium chloride is the preferred salt with cerium oxide also being exemplified. There is no disclosure of lanthanum metal oxides with a weight average particle size of from 0.5 to 50 μm.

U.S. Pat. No. 4,165,366 (Mellberg) discloses a prophylactic paste comprising an abrasive at from 40 to 60% by weight of the composition. The preferred abrasive is medium grade pumice, although other abrasives may be used such as zirconium silicate, alumina, cerium oxide, silicon carbide and the like.

GB 2 001 849 (ICI) discloses acomposition for use in cleaning teeth. Specifically it discloses water-soluble salts of lanthanum and the lanthanides. There is no disclosure of their oxides.

Despite all that is available in the prior art there remains the need for improved abrasives suitable for use in toothpastes for everyday use.

Accordingly, the present invention provides an oral composition according to claim 1.

The invention includes the use of an oxide of any of the lanthanide metals and these include La, Ce, Pr, Nd, Pm, Sm, Eu, Gd, Tb, Dy, Ho, Er, Tm, Yb and Lu. However, the most preferred are lanthanum oxide (La₂O₃) and cerium oxide (CeO₂).

Preferably the lanthanide metal oxide has a weight average particle size of from 0.5 to. 50 μm, preferably from 0.6 to 10 μm. Such particle size can be measured on a Malvern Mastersizer® according to standard protocols.

Preferably, the lanthanide metal oxide is present in the composition at from 0.01 to 5% by weight of the composition.

More preferably from 0.2 to 3% by weight and most preferably from 0.6 to 1.5% by weight of the composition.

The further abrasive in the oral composition is any abrasive known in the art of oral compositions and includes calcium carbonate, silica, dicalcium phosphates, etc.

Where the further abrasive is silica it is preferably present at from 3 to 15% by weight of the composition.

Where the further abrasive is calcium carbonate it is preferably present at from 10 to 60% by weight of the composition.

In a second aspect the invention provides for the use of from 0.01 to 5% by weight of a lanthanide metal oxide as an abrasive in an oral composition.

Preferred embodiments of the composition for this second aspect of the invention are as described in respect of the first aspect of the invention.

The oral composition according to the invention comprise further ingredients which are common in the art, such as:

antimicrobial agents, e.g. Triclosan, chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates,. insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

The oral composition according to the invention is made by standard protocols known to the person skilled in the art.

### EXAMPLE

The following experiment illustrates the surprising effect of cerium oxide and lanthanum oxide in combination with abrasive silica.

The RDA is measured according to the protocol described in A Laboratory Method for Assessment of Dentifrice Abrasivity J J Hefferren Journal Dent Res Vol 55 No 4 563-573 (1976).

The PCR is measured according to the protocol described in In vitro removal of stain with dentifrices G K Stookey, T A Burkhard and B R Schemehorn Journal Dent Res Vol 61 No 11 1236-1239 (1982).

Both the cerium oxide and lanthanum oxide are commercially available from Aldrich.

Cerium (IV) oxide powder, cat no: 21,157-5.

Lanthanum oxide, cat no: 19,992-3.

| Sample | RDA | PCR |
|---|---|---|
| Control | 88.45 | 73.18 |
| Control + 1% (w/w) | 87.3 | 90.96 |
| Cerium oxide | | |
| Control + 1% (w/w) | 84.1 | 79.45 |
| Lanthanum oxide | | |

Accordingly, it is evident that the addition of either of cerium oxide or lanthanum oxide to a standard toothpaste comprising abrasive silica results in an improved PCR without the expected increase in RDA.

1. Oral composition comprising as abrasive a mixture of a lanthanide metal oxide and a further abrasive, characterised in that the lanthanide metal oxide has a weight average particle size of from 0.5 to 50 μm.

2. Oral composition according to claim 1, wherein the further abrasive is selected from silica, calcium carbonate and mixtures thereof.

3. Oral composition according to claim 1, wherein the lanthanide metal oxide is lanthanum oxide or cerium oxide.

4. Oral composition according to claim 1, wherein the lanthanide metal oxide has a weight average particle size of from 0.6 to 10 μm.

5. Oral composition according to claim 1, wherein the further abrasive is calcium carbonate and is present at from 10 to 60% by weight of the composition.

6. Oral composition according to claim 1, wherein the further abrasive is silica and is present at from 3 to 15% by weight.

7. Use of from 0.01 to 5% by weight lanthanide metal oxide as an abrasive in an oral composition.
